(19) United States
(12) Patent Application Publication
(10) Pub. No.: US 2016/0328532 A1
(43) Pub. Date: Nov. 10, 2016

(54) SYSTEM, METHOD AND SOFTWARE PRODUCT FOR MEDICAL TELEPRESENCE PLATFORM

(71) Applicants: Anthony A. Rieder, Menomonee Falls, WI (US); Edward Rapee, III, Brookfield, WI (US)

(72) Inventors: Anthony A. Rieder, Menomonee Falls, WI (US); Edward Rapee, III, Brookfield, WI (US)

(21) Appl. No.: 15/146,456

(22) Filed: May 4, 2016

Related U.S. Application Data
(60) Provisional application No. 62/156,403, filed on May 4, 2015.

(51) Int. Cl. G06F 19/00; A47B 21/03; A47B 21/02; A61G 13/00; A61B 1/227; A61B 5/00; A61B 3/10; F16M 11/42; F16M 11/24

(52) U.S. Cl. CPC G06F 19/3418; F16M 11/42; A47B 21/0314; A47B 21/02; F16M 11/24; A61B 1/227; A61B 5/0077; A61B 3/10; A61G 13/0018; A47B 2021/0335; A47B 2021/0364; A47B 2200/0066; A47B 2031/003

(57) ABSTRACT

A system and method allows for remote specialty physicians to have the capability to remotely control what they can see through a video conferencing system as well as what they can interact with by direct control of a robotic arm that is part of a physician-side portal and a patient-side portal. Two videoconferencing sessions will be handled by the solution. This allows for the remote physician to control, from his or her end, what he or she wishes to see without impacting the local user's ability to complete their exam and share this information via the first portal. While this is being done, the remote user is interacting via the second portal all in the same virtual room or session.

This application claims the benefit and priority of U.S. Provisional Patent Application No. 62/156,403 filed May 4, 2015.

### FIELD OF THE INVENTION

This invention relates very generally to medical diagnostics and to electronic data management in the medical and healthcare industries. It also relates generally to web-based (i.e. utilized by means of the virtual community that exists on the “internet” or within the “world wide web,” or “www”) methods and systems for acquiring, storing, processing, retrieving and displaying information and data. More specifically, the present invention relates to a system, method and software product that provides a medical telepresence functionality for use with any mobile computing device, but which is particularly useful with electronic interactive “terminals,” including touch-screen devices. Further, the present invention relates to a system and method that uses an exam table, bed chair and mobile cart equipped with telepresence capabilities and wireless instruments, all of which allows a specialist physician or other healthcare provider to remotely provide patient care in hospitals, emergency departments (“ED”) and clinical program locations.

### BACKGROUND OF THE INVENTION

Community hospitals face increasing problems obtaining emergency and hospital on-call coverage from specialist physicians. The diminished willingness of specialist physicians to provide on-call coverage is occurring as hospital EDs confront an ever-increasing demand for services. Factors influencing physician reluctance to provide on-call coverage include decreased dependence on hospital admitting privileges as more services shift to non-hospital settings; payment for emergency care, especially for uninsured patients; and medical liability concerns. Hospital strategies to secure on-call coverage include enforcing hospital medical staff bylaws that require physicians to take call, contracting with physicians to provide coverage, paying physicians stipends, and employing physicians. Even with these strategies, hospitals continue to struggle with inadequate on-call coverage, which threatens patients' timely access to high-quality emergency care, reduces patient satisfaction and may contribute to raising health care costs.

Many physicians are reluctant to take call. In the past, physicians provided on-call emergency coverage in exchange for hospital admitting privileges, which allowed them to connect with new patients and helped build their practices. In addition, heavy public subsidization of medical education and residency training traditionally has been accompanied by an unwritten social contract for physicians to maintain the core competencies of their specialty in hospitals where they practice and to provide some emergency call. Hospitals enforce on-call requirements through medical staff bylaws or other contractual arrangements with physicians. With many specialists now shifting the focus of their practices away from hospital settings or to specialty hospitals that don't have EDs, they are less reliant on hospital admitting privileges to care for their patients or to maintain a practice.

Payment for emergency care, and physician services in general, is another factor in specialists' reluctance to provide on-call coverage. Many physicians believe payment for care provided while on call is inadequate, and when they are required to care for uninsured patients, the situation becomes untenable. Time spent by a physician seeing ED patients has an opportunity cost—in terms of time away from insured patients in their office practice. ED call is seen as a burden that negatively affects physician quality of life and practice finances. Hospital and emergency services provided by specialists also increases their exposure to potential medical liability.

Finally, the specialist on-call coverage issue places a disproportionate burden on physicians willing to provide coverage, increasing the potential for adverse patient outcomes as the workload increases and morale declines. As fewer physicians agree to take call, specialists who provide on-call coverage in some areas must cover multiple hospitals on the same night.

All of this ultimately results in a reduction or lack of on-call physician services. This has the potential to lead to adverse patient outcomes. In some cases, this can result in prolonged patient care and inconvenience, increased costs, and hospital transfers due to lack of covered services.

The key factors contributing to the problem that need to be considered include market changes that discourage specialist physicians from providing emergency on-call coverage, shrinking number of specialists available to provide on-call coverage services due to attrition, system alignment or employment, and the rising number of uninsured people and others seeking hospital and emergency services, which are likely to further aggravate the situation, creating additional quality and cost pressures for the health care system.

In accordance with the foregoing, these inventors have sought to answer the question: How can key on-call and available coverage services be provided to meet a growing demand, with fewer resources?

Today's world of electronic data processing and electronic communication allows individuals to manage data and assets of all sorts and in a myriad of ways. The medical arts are no exception. Accordingly, this reality goes a long way in answering the question posed above. The proliferation of personal electronic devices has enhanced the ability of users to receive, store and transmit data in a digital format, in real-time or as a recording of sorts. In the view of these inventors, and to facilitate the most efficient use of data that can be and is acquired from patient examinations and patient assessments, it would be desirable to exploit this digital functionality in combination with certain specific hard assets to allow specialist physicians to “capture” patient information and to formulate and implement a treatment plan.

All such data uploading must, however, be accomplished in a completely secure platform in order to meet the stringent privacy rights of health care patients that have been established under the Health Insurance Portability and Accountability Act of 1996 (“HIPAA”). Prior to HIPAA, no generally accepted set of security standards or general requirements for protecting patient information existed in the healthcare industry. At the same time, new technologies were evolving, and the healthcare industry began to move away from paper processes and rely more heavily on the use of electronic information systems to pay claims, answer eligibility questions, provide health information and conduct a whole host of other administrative and clinically based functions. While this means that the medical workforce can be more mobile and efficient (i.e., physicians can check patient records and test results from virtually any location and wherever they are), the rise in the adoption rate of these technologies also increases the potential for security risks. Under HIPAA, the Secretary of the U.S. Department of Health and Human Services (“HHS”) was required to develop regulations protecting the privacy and security of certain health information. To fulfill this requirement, HHS published what are commonly known as the HIPAA “Privacy Rule” and the HIPAA “Security Rule.” The “Privacy Rule,” or Standards for Privacy of Individually Identifiable Health Information, establishes national standards for the protection of certain healthcare information. The Security Standards for the Protection of Electronic Protected Health Information (the “Security Rule”) establish a national set of security standards for protecting certain healthcare information that is held or transferred in electronic form. The Security Rule operationalizes the protections contained in the Privacy Rule by addressing the technical and non-technical safeguards that organizations called “covered entities” must put in place to secure individuals' “electronically-protected healthcare information” (or “e-PHI”).

Specifically, The HIPAA Privacy Rule provides federal protections for individually identifiable health information held by covered entities and their business associates and gives patients an array of rights with respect to that information. At the same time, the Privacy Rule is balanced so that it permits the disclosure of healthcare information needed for patient care and other important purposes. To this point, it will be necessary that the telepresence platform, configured by these inventors in accordance with the present invention, meet the compliance requirements of HIPAA.

### SUMMARY OF THE INVENTION

In accordance with the foregoing, these inventors have devised a telepresence medical technology platform that can be integrated into and used with medical hardware in both stationary and mobile designs. The components of each design include the capability to provide exam information, and endoscopic exam information in particular, either in real time via a HIPAA-compliant videoconferencing solution, such as VIDYO® teleconferencing and video conferencing services (VIDYO is a registered mark of Vidyo, Inc.) or an equivalent service. Alternatively, exam information can be securely stored on the platform for reviewing and sharing at a later time. Both platforms utilize a combination of wireless, such as FIREFLY™ wireless products (FIREFLY is a mark of Firefly Global), and connected instrumentation—all for the purpose of obtaining patient biometrics, and other exam information, including endoscopic findings. Such instrumentation includes, for example, digital dermatoscopes, otoscopes and endoscopes, among others.

The present invention comprises platforms that utilize a software application to simplify the use of the platforms and allow the user to connect to remote specialist physician or health care provider support via the secured video conferencing solution. The software application will allow connection to remote on-call specialist physicians or health care providers or other telepresence networks via a DOCTORS LIVE™ (Doctors Live is a mark of Care Integrations, LLC) or other similar connections. The telepresence functionality is not limited to any particular use or location. For example, other uses in elder care, worker compensation, on-site provider clinic support and primary care walk-in clinics are all areas where the present invention could be utilized.

With the present invention, remote specialty physicians will have the capability to remotely control what they can see through the video conferencing system as well as what they can interact with by direct control of a robotic arm that is part of both platforms. Two videoconferencing sessions will be handled by the solution. This allows for the remote physician to control, from his or her end, what he or she wishes to see without impacting the local user's ability to complete their exam and share this information via a first “portal.” While this is being done, the remote user is interacting via a second “portal” all in the same “room” or session.

By way of a brief specific example, the present invention contemplates the inclusion and use of wireless instruments (such as otoscope, dermatoscope and endoscopic cameras—although the present invention is not so limited) that are typically utilized in providing ear, nose and throat (“ENT”) care. In short, and for purposes of this detailed disclosure, it is to be understood that any other medical diagnostic tool, instrument or equipment that is capable of measuring and analyzing physical parameters or body metrics (collectively “medical diagnostic tools”) can be used with the system and method of the present invention. The ENT capabilities described herein are but one usage of the system and method and presented as a non-limiting example only.

At least one fully-equipped exam chair with telepresence capabilities would be placed in a first location. This provides a “virtual” telepresent support room to be utilized by ED staff or other ENT staff when providing care to patients that may need ENT consult services. Additionally, at least one fully-equipped mobile cart with telepresence capabilities for hospital, ED or clinical program use would also be provided. Robotic arms, such as a KUBI® mounting device (KUBI is a registered mark of Revolve Robotics, Inc.), are used with the exam chair and mobile cart to support and move a “terminal.” As used in this disclosure, a “portal” comprises either the mobile cart or the exam table or chair, and a “terminal” is any interactive digital device or personal digital assistant having a touch screen, such as an iPad® brand tablet computer (iPad® is a registered mark of Apple Inc.) that is used at the portals. By using interchangeable elements or components, the present invention is intended to be modular in its construction. Lastly, the functionality of telepresence would be provided with a fully-compliant HIPPA technology platform.

The foregoing and other features of the apparatus, method and system of the present invention will become apparent from the detailed description that follows.

### BRIEF DESCRIPTION OF THE DRAWINGS

FIG. 1 is a perspective view of a movable medical data cart of the type used in the system of the present invention, which cart can serve as a first “portal” in accordance with the system and method of the present invention.

FIG. 2 is a perspective view of a patient table or chair of the type used in the system of the present invention, which table or chair can serve as a second “portal” in accordance with the system and method of the present invention.

### DETAILED DESCRIPTION

At the outset, it is to be specifically noted that there are several key innovations or functionalities that are integrated into the system, method and software product of the present invention. It is also to be generally understood that the foregoing functionalities can be applicable to various types of camera phones or “terminals,” including, but not limited to, iPhone® devices. Examples of such terminals include mobile as well as stationary terminals, including mobile phones, smart phones, computers, digital broadcast terminals, personal digital assistants (“PDA”) and portable multimedia players. Further description will be with regard to a mobile terminal, but it should be noted that such teachings apply equally to other types of terminals.

The mobile terminal in accordance with the preferred embodiment of the present invention can include a wireless communication unit, an audio/video input unit, a user input unit, a sensing unit, an output unit, a memory, an interface unit, a controller, a power supply unit and the like. It is also to be understood that implementing all of the components is not a requirement as greater or fewer components may be implemented.

The wireless communication unit typically includes one or more components which permits wireless communication between the mobile terminal and a wireless communication system or network within which the mobile terminal is located. For instance, the wireless communication unit can include a broadcast receiving module, a mobile communication module, a wireless internet module and the like. The broadcast receiving module receives a broadcast signal and/or broadcast associated information from an external broadcast managing server via a broadcast channel. The broadcast channel may include a satellite channel and a terrestrial channel. The broadcast managing server generally refers to a server which generates and transmits a broadcast signal and/or broadcast associated information or a server which is provided with a previously generated broadcast signal and/or broadcast associated information and then transmits the provided signal or information to a terminal. The broadcast associated information includes information associated with a broadcast channel, a broadcast program, a broadcast service provider, etc. The broadcast associated information can be provided via a mobile communication network. In this case, the broadcast associated information can be received by the mobile communication module. The broadcast associated information can be implemented in various forms. For instance, broadcast associated information may include an electronic program guide of digital multimedia broadcasting and electronic service guide of digital video broadcast-handheld. The broadcast receiving module may be configured to receive broadcast signals transmitted from various types of broadcast systems. The broadcast signal and/or broadcast associated information received by the broadcast receiving module may be stored in a suitable device, such as a memory. The mobile communication module transmits/receives wireless signals to/from one or more network entities (e.g., base station, external terminal, server, etc.). Such wireless signals may represent audio, video, and data according to text/multimedia message transceivers, among others. The wireless internet module supports Internet access for the mobile terminal. This module may be internally or externally coupled to the mobile terminal. In this case, the wireless Internet technology can include Wireless LAN, Wi-Fi, Wibro, Wimax, HSDPA, etc.

A position-location module could also be utilized in the present invention, the position-location module being functionally adapted to identify or otherwise obtain the location of the mobile terminal. This module may be implemented with a global positioning system (“GPS”) module.

The user input unit generates input data responsive to user manipulation of an associated input device or devices. Examples of such devices include a keypad, a dome switch, a touchpad (e.g., static pressure/capacitance), a jog wheel, a jog switch, etc.

The output unit generates outputs relevant to the senses of sight, hearing, touch and the like. Further, the output unit includes a display which is typically implemented to visually display (output) information associated with the mobile terminal. For example, if the mobile terminal is operating in a phone call mode, the display will generally provide a user interface (“UI”) or graphical user interface (“GUI”) which includes information associated with placing, conducting, and terminating a phone call. As another example, if the mobile terminal is in a video call mode or a photographing mode, the display may additionally or alternatively display images which are associated with these modes, the UI or the GUI. The display module may be implemented using known display technologies including, for example, a liquid crystal display (“LCD”), a thin film transistor-liquid crystal display (“TFT-LCD”), an organic light-emitting diode display (“OLED”), a flexible display and a three-dimensional display. Some of the above displays can be implemented in a transparent or optical transmissive type, which can be named a transparent display. Where the display and a sensor for detecting a touch action (hereinafter called “touch sensor”) configures a mutual layer structure (hereinafter called “touchscreen”), it is able to use the display as an input device as well as an output device. In this case, the touch sensor can be configured as a touch film, a touch sheet, a touchpad or the like. The touch sensor can be configured to convert a pressure applied to a specific portion of the display or a variation of a capacitance generated from a specific portion of the display to an electric input signal. Moreover, it is able to configure the touch sensor to detect a pressure of a touch as well as a touched position or size. If a touch input is made to the touch sensor, a signal corresponding to the touch is transferred to a touch controller. The touch controller processes the signal and then transfers the processed signal to the controller. Therefore, the controller is able to know whether a prescribed portion of the display is touched. A proximity sensor can be provided to an internal area of the mobile terminal enclosed by the touchscreen or around the touchscreen. The proximity sensor is the sensor that detects a presence or non-presence of an object approaching a prescribed detecting surface or an object existing around the proximity sensor using an electromagnetic field strength or infrared ray without mechanical contact. Hence, the proximity sensor has durability longer than that of a contact type sensor and also has utility wider than that of the contact type sensor. The proximity sensor can include one of a transmissive photoelectric sensor, a direct reflective photoelectric sensor, a mirror reflective photoelectric sensor, a radio frequency oscillation proximity sensor, an electrostatic capacity proximity sensor, a magnetic proximity sensor, an infrared proximity sensor and the like. In case that the touchscreen includes the electrostatic capacity proximity sensor, it is configured to detect the proximity of a pointer using a variation of electric field according to the proximity of the pointer. In this case, the touchscreen can be classified as the proximity sensor.

The memory unit is generally used to store various types of data to support the processing, control, and storage requirements of the mobile terminal. Examples of such data include program instructions for applications operating on the mobile terminal, contact data, phonebook data, messages, audio, still pictures, moving pictures, etc. Further, a recent use history or a cumulative use frequency of each data (e.g., use frequency for each data set) can be stored in the memory unit. The memory may be implemented using any type or combination of suitable volatile and non-volatile memory or storage devices including hard disk, random access memory (“RAM”), static random access memory (“SRAM”), electrically erasable programmable read-only memory (“EEPROM”), erasable programmable read-only memory (“EPROM”), programmable read-only memory (“PROM”), read-only memory (“ROM”), magnetic memory, flash memory, magnetic or optical disk, multimedia card micro type memory, card-type memory (e.g., SD memory, XD memory, etc.), or other similar memory or data storage device. The mobile terminal is able to operate in association with a web storage for performing a storage function of the memory on the internet.

The interface unit is often implemented to couple the mobile terminal with external devices. The interface unit receives data from the external devices or is supplied with the power and then transfers the data or power to the respective elements of the mobile terminal or enables data within the mobile terminal to be transferred to the external devices. The interface unit may be configured using a wired/wireless headset port, an external charger port, a wired/wireless data port, a memory card port, a port for coupling to a device having an identity module, audio input/output ports, video input/output ports, an earphone port and/or the like.

The controller typically controls the overall operations of the mobile terminal. For example, the controller performs the control and processing associated with voice calls, data communications, video calls, etc. The controller may include a multimedia module that provides multimedia playback. The multimedia module may be configured as part of the controller, or implemented as a separate component. The controller is able to perform a pattern recognizing process for recognizing a writing input and a picture drawing input carried out on the touchscreen as characters or images, respectively.

It should also be understood that various embodiments described herein may be implemented in a computer-readable medium using, for example, computer software, hardware, or some combination thereof. For a hardware implementation, the embodiments described herein may be implemented within one or more application specific integrated circuits (“ASIC”), digital signal processors (“DSP”), digital signal processing devices (“DSPD”), programmable logic devices (“PLD”), field programmable gate arrays (“FPGA”), processors, controllers, micro-controllers, microprocessors, other electronic units designed to perform the functions described herein, or a selective combination thereof. Such embodiments may also be implemented by the controller.

For a software implementation, the embodiments described herein may be implemented with separate software modules, such as procedures and functions, each of which perform one or more of the functions and operations described herein. Software implementation can be accomplished via software that is installed directly on a terminal or may be provided as a software as a service (“SAAS”) format. The software codes can be implemented with a software application written in any suitable programming language and may be stored in memory such as the memory, and executed by a controller or processor, such as the controller. That is, the software includes source code which is a list of instructions, written in a selected computer language, and then converted into computer machine language, which language the computer uses to build the software “machine” described by the instructions. The software machine is made up of the components referred to above. The source code is a detailed “blueprint” telling the computer how to assemble those components into the software machine. Further, the source code is organized into separate files, files are organized into separate modules, and modules are organized into separate functions or routines to accomplish, via pre-programmed algorithms, the necessary steps in accordance with the method and system of the present invention. It is to be understood that the specific way that the source code is organized into files, modules and functions is a matter of programmer design choice and is not a limitation of the present invention. It should also be understood that the present invention is made possible by virtue of the existence of the internet.

Lastly, the power supply unit provides power required by the various components for the mobile terminal. The power may be provided internally, externally or a combination thereof.

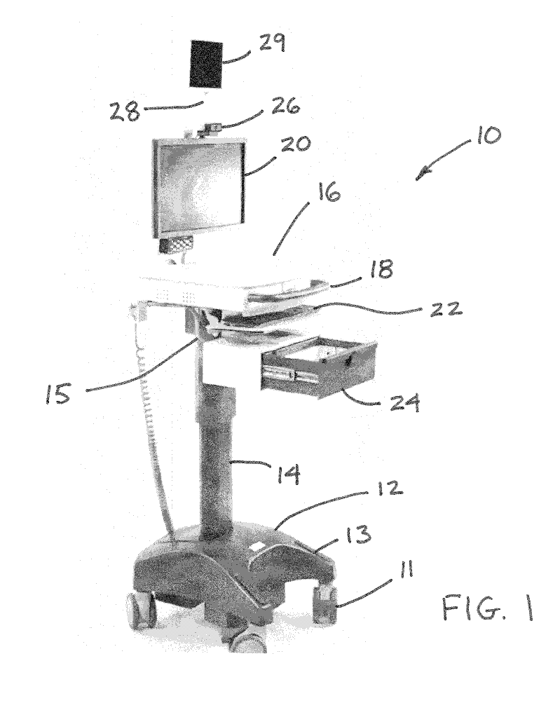

In accordance with the foregoing, it is to be specifically noted again that there are several key innovations contemplated in the method, system and software product of the present invention. Specifically, and referring first to FIG. 1, it will be seen that a mobile cart, generally identified 10, such as a DataCart™ DCT-6 workstation (DataCart is a mark of Lund Industries), is provided. It is to be understood, however, that the cart 10 is not limited to that described herein. This cart 10 can also be referred to herein as the “first portal.” As shown, cart 10 comprises a base 12 and a height-adjustable vertical support member 14, the support member 14 being extendable from a “sitting” height to a “standing” height. The base 12 further comprises castors 11 and footrests 13 for ergonomic purposes. The base 12 also comprises a rapid recharge on-board battery (not shown) which is used to provide power to the cart 10 and its various electronic components as needed.

At the uppermost portion 15 of the support member 14 is a work surface 16, the work surface 16 comprising a handle 18 to allow the user to grasp the handle 18 and move the cart 10 as desired or required. Below the work surface 16 is a pull-out keyboard support 22 and a service drawer 24. Above the work surface 16 is a monitor 20 in the form of an LED high resolution touch screen. A camera 26 is also provided together with a lockable robotic head 28 with a PDA 29 having a screen, although the audio-visual component could be built into PDA 29. The monitor 20 is used to access, display and enter patient data of other information. The PDA 29 is a terminal which includes the display screen to allow the user of the cart 10 to view a patient at a remote location and to audibly and visually interact with that patient. The cart 10 can also comprise a wireless otoscope, dermatoscope and wired iris scope (also not shown). The cart 10, however, is not limited to those specific equipment options. For purposes of this detailed disclosure, it is to be understood that any other medical diagnostic tool, instrument or equipment that is capable of measuring and analyzing physical parameters or body metrics (again, collectively “medical diagnostic tools”) can be used with the system and method of the present invention. The cart 10 does include an on-board computer which enables the physician-side of the telepresence software of the present invention.

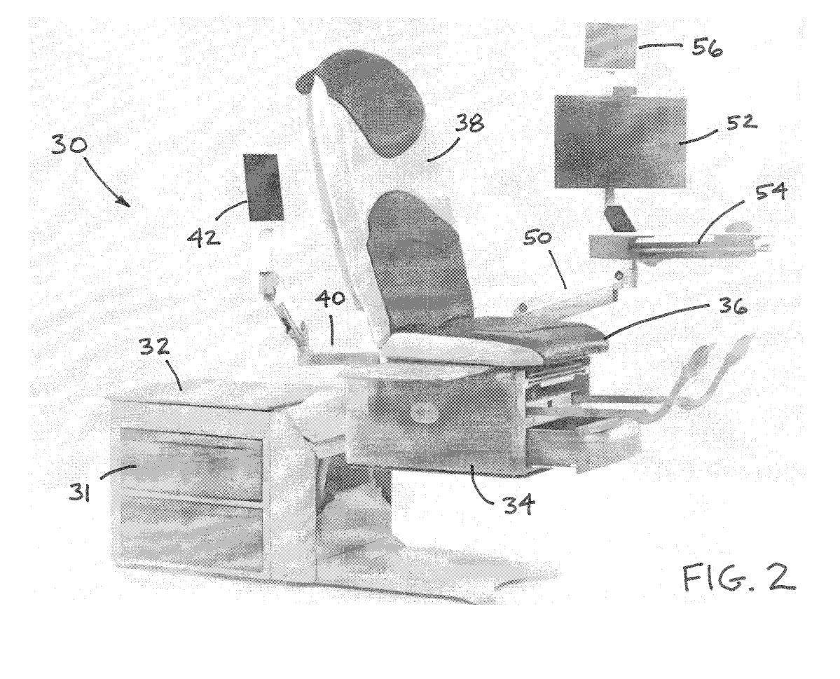

Referring now to FIG. 2, it shows a medical examination table, generally identified 30, of the type that is also used in the system of the present invention. This table 30 can also be referred to herein as the “second portal.” The table 30 is preferably of the type disclosed in U.S. Pat. No. 8,479,329 to DeBraal, et al., issued Jul. 19, 2013. It is to be understood, however, that the precise table type is not a limitation of the present invention. The medical examination table 30 comprises a floor base 32 having a storage area 31, a patient support base 34 which is movable between a lowered position and a raised position independent of the floor base 32, and a lift mechanism (not shown) coupled to the patient support base 34 for moving the patient support base 34 between the lowered position and the raised position. This allows a patient to transfer or move onto a seat 36 either from a wheel chair (not shown) or from an ambulatory standing position.

The patient support base 34 further comprises a reclining backrest 38. Movement of the patient support base 34 independent or separately from the floor base 32 and without interfering with the storage areas 31 within the floor base 32 allows for the efficient use of the examination table 30 as a storage area.

The examination table 30 also comprises, to one side of the patient sitting area, a first lockable robotic arm and head 40 with a PDA 42 having a screen. To the other side of the patient sitting area, a monitor 52 in the form of an LED high resolution touch screen is provided. The monitor 52 is used to access, display and enter patient data of other information and is mounted to a second robotic arm 50 having a keyboard 54 and a PDA 56 of its own. The PDA 56 is a terminal which includes the display screen to allow the patient to visually and audibly interact with a specialty physician at a remote location, such as where the first portal 10 is located.

The table 30 can also comprise a wireless otoscope, dermatoscope and wired iris scope (not shown) or other medical diagnostic tools, as that term has been previously defined. That is, the table 30 is not limited to any specific diagnostic equipment or to any one type of specific equipment options. The table 30 also includes an on-board computer which enables the patient-side of the telepresence software of the present invention. It should also be mentioned that the table 30 of the present invention can include fully customizable ENT, cardiac, psychiatric, ultrasound and other mobile clinic conversions (also not shown). The table 30 is not limited to those particular conversions and others are considered to be included within the scope of the present invention.

Lastly, it is to be understood that the present invention comprises software that provides a HIPAA-compliant videoconferencing solution, such as the previously mentioned VIDYO® teleconferencing and video conferencing services or an equivalent service. It also utilizes a combination of wireless functionality (including Wi-Fi functionality) and connected instrumentation—all for the purpose of obtaining patient biometrics, and other exam information, including endoscopic findings.

The details of the invention having been disclosed in accordance with the foregoing, we claim:

1. A system for providing a healthcare professional with the ability to render telemedicine services to a patient, the system comprising:
a mobile cart, the cart comprising:
a base;
a height-adjustable vertical support member;
a work surface; and
a pull-out keyboard support;
an onboard computer which enables the healthcare professional-side of the telemedicine services;
a monitor;
a camera;
a lockable robotic head; and
a PDA;
all of the foregoing enabling the healthcare professional-side of the telemedicine services to be provided via visual and audible interaction with a patient at a remote location.

2. The system of claim 1 wherein the support member is extendable from a sitting height to a standing height.

3. The system of claim 1 wherein the base comprises castors and footrests.

4. The system of claim 1 wherein the base comprises a rapid recharge on-board battery.

5. The system of claim 1 wherein the monitor is a high resolution LED touchscreen, the monitor being used to access, display and enter patient data or other information.

6. The system of claim 1 further comprising other medical diagnostic tools.

7. The system of claim 1 further comprising:
a medical examination table, the table comprising:
a floor base;
a patient support base; and
a patient seat;
an onboard computer which enables the patient-side of the telemedicine services;
a first lockable robotic arm;
a PDA attached to the first lockable robotic arm;
a second lockable robotic arm, the second lockable robotic arm comprising:
a keyboard;
a monitor; and
a PDA;
all of the foregoing enabling the patient-side of the telemedicine services to be provided via visual and audible interaction with a healthcare professional at a remote location.

8. The system of claim 7 wherein the monitor of the second robotic arm is a high resolution LED touchscreen, the monitor being used to access, display and enter patient data or other information.

9. The system of claim 7 further comprising other medical diagnostic tools.

10. The system of claim 7 wherein the on-board computers utilize telepresence software to enable the rendering of telemedicine services.

11. A system for providing a healthcare professional with the ability to render telemedicine services to a patient, the system comprising a first portal and a second portal, the first portal comprising:
a mobile cart, the cart comprising:
a base;
a height-adjustable vertical support member;
a work surface; and
a pull-out keyboard support;
an onboard computer which enables the healthcare professional-side of the telemedicine services;
a monitor;
a camera;
a lockable robotic head; and
a PDA;
the first portal enabling the healthcare professional-side of the telemedicine services to be provided via visual and audible interaction with a patient at a remote location; and
the second portal comprising:
a medical examination table, the table comprising:
a floor base;
a patient support base; and
a patient seat;
an onboard computer which enables the patient-side of the telemedicine services;
a first lockable robotic arm;
a PDA attached to the first lockable robotic arm;
a second lockable robotic arm, the second lockable robotic arm comprising:
a keyboard;
a monitor; and
a PDA;
the second portal enabling the patient-side of the telemedicine services to be provided via visual and audible interaction with a healthcare professional at a remote location.

12. The system of claim 10 wherein the monitors are high resolution LED touchscreens, the monitors being used to access, display and enter patient data or other information.

13. The system of claim 10 further comprising other medical diagnostic tools.

14. The system of claim 10 wherein the on-board computers utilize telepresence software to enable the first and second portals.

15. A method of using a first portal and a second portal to provide a healthcare professional with the ability to render telemedicine services to a patient, the method comprising the steps of:
providing a first portal, the first portal comprising a mobile cart having a base, a height-adjustable vertical support member, a work surface and a pull-out keyboard support; the first portal further comprising an onboard computer which enables the healthcare professional-side of the telemedicine services, a monitor, a camera, a lockable robotic head, and a PDA;
enabling the healthcare professional-side of the telemedicine services to use the first portal to visually and audibly interaction with a patient at a remote location;
providing a second portal, the second portal comprising a medical examination table, the table comprising a floor base, a patient support base and a patient seat; the second portal further comprising an onboard computer which enables the patient-side of the telemedicine services, a first lockable robotic arm, a PDA attached to the first lockable robotic arm, a second lockable robotic arm, the second lockable robotic arm comprising a keyboard, a monitor and a PDA; and
enabling the patient-side of the telemedicine services to be provided via visual and audible interaction with a healthcare professional at a remote location.

16. The method of claim 15 wherein the monitors provided are high resolution LED touchscreens, the monitors being used to access, display and enter patient data or other information.

17. The method of claim 15 further comprising the step of providing other medical diagnostic tools.

18. The method of claim 15 wherein the on-board computers utilize telepresence software to enable the first and second portals.
